(19) United States
(12) Patent Application Publication
(10) Pub. No.: US 2006/0173417 A1
(43) Pub. Date: Aug. 3, 2006

(54) Combination insulin pen and transmission device

(76) Inventors: Richard Rosen, Belmont, MA (US); Ian Hunter, Lincoln, MA (US)

Correspondence Address: GREENBERG TRAURIG, LLP, MET LIFE BUILDING, 200 PARK AVENUE, NEW YORK, NY, 10166, US

(21) Appl. No.: 11/072,178

(22) Filed: Mar. 4, 2005

Related U.S. Application Data
(60) Provisional application No. 60/642,845, filed on Jan. 10, 2005.

(51) Int. Cl. A61M 5/00

(52) U.S. Cl. 604/187

(57) ABSTRACT

Various embodiments of the present invention are directed to a combination insulin pen and transmission device. In one specific embodiment an insulin injection device is provided, comprising: a container which holds the insulin; and a data transmitter, which data transmitter transmits data regarding injection of the insulin; wherein the data regarding injection of the insulin includes (but is not limited to), for at least one injection, at least one of a dosage amount, a time of injection and a date of injection. In another specific embodiment an insulin injection and data tracking system is provided, comprising: a portable device including: (a) a container which holds the insulin; and (b) a data transmitter, which data transmitter transmits data regarding injection of the insulin; and a data receiver, which data receiver receives the transmitted data; wherein the data regarding injection of the insulin includes (but is not limited to), for at least one injection, at least one of a dosage amount, a time of injection and a date of injection.

### RELATED APPLICATIONS

This application claims the benefit of U.S. Provisional Application Ser. No. 60/642,845, filed Jan. 10, 2005

### FIELD OF THE INVENTION

Various embodiments of the present invention are directed to a combination insulin pen and transmission device.

In one specific embodiment an insulin injection device is provided, comprising: a container which holds the insulin; and a data transmitter, which data transmitter transmits data regarding injection of the insulin; wherein the data regarding injection of the insulin includes (but is not limited to), for at least one injection, at least one of a dosage amount, a time of injection and a date of injection.

In another specific embodiment an insulin injection and data tracking system is provided, comprising: a portable device including: (a) a container which holds the insulin; and (b) a data transmitter, which data transmitter transmits data regarding injection of the insulin; and a data receiver, which data receiver receives the transmitted data; wherein the data regarding injection of the insulin includes (but is not limited to), for at least one injection, at least one of a dosage amount, a time of injection and a date of injection.

### BACKGROUND OF THE INVENTION

Insulin pens are devices that can provide an easy way to carry and give insulin (e.g., when away from home). Also, for people who are not comfortable working with syringes and bottles to draw up and measure individual doses, the pens provide a convenient and accurate alternative. There are two types of conventional insulin pens: those that are completely disposable after providing a single dose of insulin; and those that use disposable cartridges to provide multiple doses. An insulin pen typically has three components: a base which contains the mechanism for measuring the correct dose; a holder for the cartridge of insulin; and a cap, which is removed and replaced by the needle prior to the injection.

### DETAILED DESCRIPTION OF THE INVENTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Further, any figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention.

In one embodiment an insulin injection device is provided, comprising: a container which holds the insulin; and a data transmitter, which data transmitter transmits data regarding injection of the insulin; wherein the data regarding injection of the insulin includes (but is not limited to), for at least one injection, at least one of a dosage amount, a time of injection and a date of injection.

In one example, the data regarding injection of the insulin may include (but not be limited to), for each of a plurality of injections, at least one of a dosage amount, a time of injection and a date of injection.

In another example, at least some of the data may be transmitted by the data transmitter at a time essentially contemporaneous with the time of each injection.

In another example, the device may further comprise a memory, wherein at least some of the data maybe stored in the memory at a time essentially contemporaneous with the time of each injection and wherein at least some of the stored data may be transmitted by the data transmitter at a later time.

In another example, the data transmitter may be wireless.

In another example, the data transmitter may be powered by a battery.

In another example, the data transmitter may be powered by a photovoltaic cell.

In another example, the data transmitter may be powered by a battery which may be charged by a photovoltaic cell.

In another example, the device may further include a needle, which needle may have a first end operatively connected to the container for receiving insulin therefrom and a second end for injecting the insulin into a user of the device.

In another example, the needle may be removable.

In another example, the device may further comprise a case.

In another example, the container may comprise a cartridge disposed within the case.

In another example, the cartridge may be replaceable.

In another embodiment an insulin injection and data tracking system is provided, comprising: a portable device including: (a) a container which holds the insulin; and (b) a data transmitter, which data transmitter transmits data regarding injection of the insulin; and a data receiver, which data receiver receives the transmitted data; wherein the data regarding injection of the insulin includes (but is not limited to), for at least one injection, at least one of a dosage amount, a time of injection and a date of injection.

In one example, the data regarding injection of the insulin may include (but not be limited to), for each of a plurality of injections, at least one of a dosage amount, a time of injection and a date of injection.

In another example, at least some of the data may be transmitted by the data transmitter to the data receiver at a time essentially contemporaneous with the time of each injection.

In another example, the portable device may further comprise a memory, wherein at least some of the data may be stored in the memory at a time essentially contemporaneous with the time of each injection and wherein at least some of the stored data may be transmitted by the data transmitter to the data receiver at a later time.

In another example, the data transmitter and the data receiver may be wireless.

In another example, the data transmitter may be powered by a battery.

In another example, the data transmitter may be powered by a photovoltaic cell.

In another example, the data transmitter may be powered by a battery which may be charged by a photovoltaic cell.

In another example, the portable device may further comprise a needle, which needle may have a first end operatively connected to the container for receiving insulin therefrom and a second end for injecting the insulin into a user of the device.

In another example, the needle may be removable.

In another example, the portable device may further comprise a case.

In another example, the container may comprise a cartridge disposed within the case.

In another example, the cartridge may be replaceable.

In another example, the data receiver may be essentially non-portable.

In another example, the data receiver may include a data storage mechanism.

In another example, the data receiver may include a data analysis mechanism.

Of note, certain embodiments of the present invention may combine the insulin delivery elements of an essentially conventional insulin pen with a transmitting device so that dosages may be electronically recorded for an individual user. In one embodiment of the present invention, the invention does not require a user to manually or otherwise record data (such as measurements) taken from the pen. Instead, data from the device may be transported by a means of communication—such as by wireless technology—to the Data Managing Device (“DMD”) of the present invention. In one embodiment of the present invention, the DMD consists of a central server apparatus (e.g., placed in relative close proximity to the user) which collects the information from the insulin pen. Collection of data by the present invention may occur without direct physical connection (e.g., wirelessly). In yet another embodiment, the transmission from the device may occur at predetermined times (e.g. download).

In another embodiment, the DMD of the invention collects data generated by the device by means of media transmission/communication, such as the user's cable, land, or cellular telephone line (at periodic intervals a connection to a Private Network Center (“PNC”) may be made to provide data from the device).

In a further embodiment, a combination insulin pen and transmitting device has a memory device within the pen so that, when the pen is not capable of transmitting data, the memory device stores the data in the pen so that the data can be later downloaded. In yet another embodiment, the stored and/or transmitted data includes, but is not limited to, the amount of insulin dosage and the specific date and time of the injection.

In yet another embodiment, the present invention is comprised of a PNC at which data is recorded, stored and/or analyzed. Data and information may be integrated with lifestyle data as well as extrinsically generated information on a patient's medical treatment. In an embodiment of the present invention, a flow of information from the device to the PNC is produced.

In one example, information from the device of the present invention may be recorded, stored and/or analyzed. In another embodiment, data from the device may be integrated with additional data (e.g., lifestyle data and/or doctor-generated information).

In a further embodiment, a combination pen and transmitting device may be powered by a photovoltaic cell and a device to store the energy (e.g., a battery). In yet another embodiment, data from the device is transported by a means of communication such as, for example, “blue tooth” technology.

In one example (which example is intended to be illustrative and not restrictive), a combination pen and transmitting device may deliver in 2-unit increments with a maximum dose of 36 units possible. In another example (which example is intended to be illustrative and not restrictive), the device may take 150-unit cartridges. In another example (which example is intended to be illustrative and not restrictive), the device may deliver in 1-unit increments with a maximum dose of 40 units (in this example the device may again take a 150-unit cartridge). In another example (which example is intended to be illustrative and not restrictive), the device may deliver in 1-unit increments but can deliver up to a 70-unit dose (in this example the device may take a larger 300-unit cartridge). In another example (which example is intended to be illustrative and not restrictive), the device may deliver insulin in single increments and deliver up to a 30-unit dose (in this example the device may take a 150-unit cartridge). Some or all of the data may be automatically transported and/or recorded to a data storage unit.

In another embodiment, a combination pen and transmitting device is disposable. For example (which example is intended to be illustrative and not restrictive), you could set the dose by turning the top half of the pen in the opposite direction from the bottom half, while the cap is still on the pen. It may click as you turn, and you could count up the dose (e.g., by 2's). While you are counting up the dose, the end of the pen opposite the needle end (where you would push to extend or retract a ball-point pen) may come out slightly for each 2 units. When you have clicked up the correct dose, you could remove the cap, screw on a disposable needle, push the needle through your skin, and inject by pressing the end of the pen. You could then remove the needle, put the cap back on, and put your pen away. When the pen is empty (in one example (which example is intended to be illustrative and not restrictive), each pen may hold 150 units), you could just throw it away and start using a new one. Of course, in other embodiments some or all of the parts of the device may be reusable (e.g., the data transmitter, memory and/or the power source). Some or all of the data may be automatically transported and/or recorded to a data storage unit.

In a further embodiment, a non-disposable combination pen and transmitting device may be loaded with insulin contained in a cartridge. For example (which example is intended to be illustrative and not restrictive), the cartridge may be made of glass, about 2 ¼″ long, 5/16″ in diameter, and may look a little like an ink cartridge for an old-fashioned cartridge pen. Each cartridge may hold, for example, 150 units, and they may be sold in packages (e.g., of 6—typically for about the price of a vial of insulin). The end of the cartridge where the needle is applied (when it's in the pen) may have a small metal ring and rubber stopper, just like a tiny insulin bottle top. The other end may be open and my have a movable rubber stopper that slides forward as insulin is dispensed from the cartridge (since the space inside gets smaller when insulin is dispensed, you don't have to inject air before each use.) The cartridge goes inside the pen, with the metal-ring end toward the front of the pen, and a plunger inside the pen resting on the movable rubber stopper in the back of the cartridge. You could set the dose by turning a ring on the end of the pen opposite the needle end. This may set how far the plunger is going to move inside the pen

The combination pen and transmitting device of the present invention may be easy to carry and to give pre-meal injections (e.g., may be easier than carrying vials and syringes). Also, it may not be as obvious as vials and syringes (therefore less embarrassing to use in public). For example, injections with a pen may be done one-handed, (e.g. under a restaurant table) counting the clicks to count the dose.

As mentioned above, the cartridge may go inside the pen, with the metal-ring end toward the front of the pen, and a plunger inside the pen resting on the movable rubber stopper in the back of the cartridge. You may set the dose by turning a ring on the end of the pen opposite the needle end. This may set how far the plunger is going to move inside the pen. Then you may screw a disposable needle onto the end of the pen and give your injection by pressing the end of the pen, or a movable clip on the side. Then you could remove the needle, re-set the dose to 0, and put everything away (thus, the pen may provide for the injection of insulin into the subcutaneous tissue between the top layer of the skin, the dermis, and the underlying muscle layer).

While a number of embodiments of the present invention have been described, it is understood that these embodiments are illustrative only, and not restrictive, and that many modifications may become apparent to those of ordinary skill in the art. For example, any method steps may be performed in any desired order, one or more steps may be deleted and/or one or more steps may be added.

1. An insulin injection device, comprising:
a container which holds the insulin; and
a data transmitter, which data transmitter transmits data regarding injection of the insulin;
wherein the data regarding injection of the insulin includes, for at least one injection, at least one of a dosage amount, a time of injection and a date of injection.

2. The device of claim 1, wherein the data regarding injection of the insulin includes, for each of a plurality of injections, at least one of a dosage amount, a time of injection and a date of injection.

3. The device of claim 2, wherein at least some of the data is transmitted by the data transmitter at a time essentially contemporaneous with the time of each injection.

4. The device of claim 2, further comprising a memory, wherein at least some of the data is stored in the memory at a time essentially contemporaneous with the time of each injection and wherein at least some of the stored data is transmitted by the data transmitter at a later time.

5. The device of claim 1, wherein the data transmitter is wireless.

6. The device of claim 1, wherein the data transmitter is powered by a battery.

7. The device of claim 1, wherein the data transmitter is powered by a photovoltaic cell.

8. The device of claim 1, wherein the data transmitter is powered by a battery which is charged by a photovoltaic cell.

9. The device of claim 1, further comprising a needle, which needle has a first end operatively connected to the container for receiving insulin therefrom and a second end for injecting the insulin into a user of the device.

10. The device of claim 9, wherein the needle is removable.

11. The device of claim 1, further comprising a case.

12. The device of claim 11, wherein the container comprises a cartridge disposed within the case.

13. The device of claim 12, wherein the cartridge is replaceable.

14. An insulin injection and data tracking system, comprising:
a portable device including: (a) a container which holds the insulin; and (b) a data transmitter, which data transmitter transmits data regarding injection of the insulin; and
a data receiver, which data receiver receives the transmitted data;
wherein the data regarding injection of the insulin includes, for at least one injection, at least one of a dosage amount, a time of injection and a date of injection.

15. The system of claim 14, wherein the data regarding injection of the insulin includes, for each of a plurality of injections, at least one of a dosage amount, a time of injection and a date of injection.

16. The system of claim 15, wherein at least some of the data is transmitted by the data transmitter to the data receiver at a time essentially contemporaneous with the time of each injection.

17. The system of claim 15, wherein the portable device further comprises a memory, wherein at least some of the data is stored in the memory at a time essentially contemporaneous with the time of each injection and wherein at least some of the stored data is transmitted by the data transmitter to the data receiver at a later time.

18. The system of claim 14, wherein the data transmitter and the data receiver are wireless.

19. The system of claim 14, wherein the data transmitter is powered by a battery.

20. The system of claim 14, wherein the data transmitter is powered by a photovoltaic cell.

21. The system of claim 14, wherein the data transmitter is powered by a battery which is charged by a photovoltaic cell.

22. The system of claim 14, wherein the portable device further comprises a needle, which needle has a first end operatively connected to the container for receiving insulin therefrom and a second end for injecting the insulin into a user of the device.

23. The system of claim 22, wherein the needle is removable.

24. The system of claim 14, wherein the portable device further comprises a case.

25. The system of claim 24, wherein the container comprises a cartridge disposed within the case.

26. The system of claim 25, wherein the cartridge is replaceable.

27. The system of claim 14, wherein the data receiver is essentially non-portable.

28. The system of claim 14, wherein the data receiver includes a data storage mechanism.

29. The system of claim 28, wherein the data receiver includes a data analysis mechanism.
